(19) United States
(12) Patent Application Publication
(10) Pub. No.: US 2008/0227208 A1
(43) Pub. Date: Sep. 18, 2008

(54) Devices and Methods for Detection of Occult Blood

(76) Inventors: Hsiao-Ching Yee, Mill Creek, WA (US); Kuo-Ching Yee, Mill Creek, WA (US); Hsian-Pei Yee, Mill Creek, WA (US)

Correspondence Address: Venture Pacific Law, PC, 5201 Great America Parkway, Suite 270, Santa Clara, CA, 95054, US

(21) Appl. No.: 11/687,476

(22) Filed: Mar. 16, 2007

(51) Int. Cl. G01N 33/72

(52) U.S. Cl. 436/66; 422/68.1

(57) ABSTRACT

The present invention relates to devices and methods for the detection of occult blood in a test sample. These devices and methods can detect the presence of one or more of the following components of occult blood: hemoglobin, transferrin, hemoglobin-haptoglobin complex, and albumin. Methods for the detection of occult blood in a test sample include the following steps: exposing said test sample to two or more of the following antibodies: anti-hemoglobin antibodies, anti-hemoglobin-haptoglobin-complex antibodies, anti-transferrin antibodies, and anti-albumin antibodies or one or more of the following antibodies: anti-hemoglobin-haptoglobin-complex antibodies, anti-transferrin antibodies, and anti-albumin antibodies; determining the level of reactions between the antibodies and their corresponding components of occult blood that may be in the test sample; and deciding on the presence of occult blood. The devices of this invention can have two or more of test areas containing two or more of the following antibodies: anti-hemoglobin antibodies, anti-transferrin antibodies, anti-hemoglobin-haptoglobin complex antibodies, and anti-albumin antibodies or one or more of test areas containing one or more of the following antibodies: anti-transferrin antibodies, anti-hemoglobin-haptoglobin complex antibodies, and, anti-albumin antibodies. The devices and methods of this invention are simple and produce rapid responses.

### FIELD OF INVENTION

This invention relates to devices and devices for the detection of occult blood. Particularly, it relates to devices and methods for the detection of the hemoglobin, transferrin, and albumin in occult blood.

### BACKGROUND

Colorectal cancer is the second most common cause of cancer-related deaths in the western world. Screening for early detection of this disease can reduce its mortality rates. It can also result in the reduction of the incidence of this disease by allowing for the detection and removal of adenomatous polyps. Currently, methods for clinical colorectal cancer screening include fecal occult blood testing, sigmoidoscopy, colonoscopy and double-contrast barium enema with fecal occult blood testing being the most commonly performed tests.

One of the symptoms of colorectal cancer or adenomatous polyps is bleeding which may lead to occult blood in the feces. Occult blood is blood present in such small quantities that it can be detected only by chemical or other tests for the blood or by microscopic or spectroscopic examination. In 1864, Van Deen first developed a chemical method, the guaiac method, to detect occult blood. Since then, numerous researchers and practitioners have improved on this guaiac method to diagnose colorectal cancer and other diseases. However, these methods for detecting occult blood have many limitations. These chemically based guaiac methods determine the presence of occult blood by the detection of the perioxidase activity of the hemoglobin in the blood. Since a peroxidase activity is also found in meats and vegetables, in order to produce accurate results, these tests require restriction of the intake of certain foods, drugs, vitamins, and other substances prior to and during the sample collection period. See U.S. Pat. No. 4,683,197. More importantly, these methods fail to identify many patients with colorectal cancer or adenoma as the sensitivity of the chemical based guaiac methods is poor, only between 26% and 40%.

In 1974, Adams first developed an immunological based method for the detection of occult blood that utilized an antigen-antibody reaction between human hemoglobin and anti-human hemoglobin antibody. See Adams: Ann. Clin. Lab. Sci., 4, 343 (1974). However, this method is complex and its sensitivity is low. Moreover, the titer and affinity of the anti-hemoglobin antibody in this method is usually so low that its takes a long time before the reaction between hemoglobin and anti-hemoglobin antibody can produce detectable results. Later test methods combining the development of latex immunochemical agglutination by Adams et al in 1974, the single radial immune diffusion (SRID) by Songster et al in 1980, and reverse-passive hemagglutination (RPHAA) by H. Selto et al in 1984 are more sensitive to low concentrations of human hemoglobin in feces. In 1990, Okuda; Shoji disclosed in their patent (U.S. Pat. No. 4,920,045) another immunological method for the detection of hemoglobin and transferrin by applying a glycosidase type bacteriolytic enzymes to a fecal sample. The enzymes release hemoglobin from its conjunction of viscous components secreted from digestive organs and cell wall components by intestinal bacteria, and prevent the decomposition of hemoglobin by intestinal bacteria. This method of detection is again complex, takes a long time, and requires the inconvenient collection of a large test sample. Usually, 2.5 to 5 grams of feces is needed in order to have sufficient hemoglobin to obtain a true positive result. The above described immunologically based test has been named the ELISA test.

Despite these improvements, clinical studies on currently used guaiac-based methods suggest that test results are positive in only about 26% to 40% of patients with colorectal cancers and only 25% to 35% of patients with polyps.

Immunological based tests have been shown to have a lower rate of false-positive results than the chemically based guaiac methods. In general, chemical based detection methods have a lower sensitivity level than immunologically based detection methods. The sensitivity of specificity of guaiac-based test is about 26% and 40% while the sensitivity for immunologically based ELISA tests for hemoglobin is between 40% and 70%.

There are now several fecal occult blood test kits in utilizing the “rapid test” method, an immunochemical test, for the detection of hemoglobin in occult blood. These test kits use an animal antibody for hemoglobin to react with a sample to test for the presence of hemoglobin in the sample. The components for testing are lyophilized on membrane papers to allow for a rapid and easy test procedure. These kits are simple to use and can rapidly detect the presence of human hemoglobin at or above 50 ng/ml. However, since hemoglobin from bleeding of the relatively upper digestive tract may be destroyed or damaged, these test kits still produce a significant number of false negatives as they lack the sensitivity to detect bleeding from polyps or colorectal cancer in the relatively upper digestive tract.

Due to the limitations of the prior art, it is therefore desirable to have novel devices and novel methods for the rapid and accurate detection of occult blood in samples in order to facility the early diagnosis and treatment of diseases that cause gastrointestinal bleeding.

### SUMMARY OF INVENTION

An object or this invention is to provide simple methods and devices for the rapid and accurate detection of the presence of occult blood.

The present invention relates to devices and methods for the detection of occult blood in a test sample. These devices and methods can detect the presence of one or more of the following components of occult blood: hemoglobin, transferrin, hemoglobin-haptoglobin complex, and albumin. Methods for the detection of occult blood in a test sample include the following steps: exposing said test sample to two or more of the following antibodies: anti-hemoglobin antibodies, anti-hemoglobin-haptoglobin-complex antibodies, anti-transferrin antibodies, and anti-albumin antibodies or one or more of the following antibodies: anti-hemoglobin-haptoglobin-complex antibodies, anti-transferrin antibodies, and anti-albumin antibodies; determining the level of reactions between the antibodies and their corresponding components of occult blood that may be in the test sample; and deciding on the presence of occult blood. The devices of this invention can have two or more of test areas containing two or more of the following antibodies: anti-hemoglobin antibodies, anti-transferrin antibodies, anti-hemoglobin-haptoglobin complex antibodies, and anti-albumin antibodies or one or more of test areas containing one or more of the following antibodies: anti-transferrin antibodies, anti-hemoglobin-haptoglobin complex antibodies, and, anti-albumin antibodies.

An advantage of this invention is that the devices and methods of this invention are simple, accurate, and is able to determine the presence of occult blood rapidly.

### DESCRIPTION OF DRAWINGS

The foregoing and other objects, aspects and advantages of the invention will be better understood from the following detailed description of preferred embodiments of this invention when taken in conjunction with the accompanying drawings in which:

FIG. 1a is an illustration of an embodiment of a detection device of this invention.

FIG. 1b is an illustration of the antigen-antibody reactions in an embodiment of a detection device of this invention.

FIG. 1c is an illustration of the antigen-antibody reactions in an embodiment of a detection device of this invention.

FIG. 2 is an illustration of a cross-sectional view of an embodiment of a detection device of this invention.

FIG. 3 is an illustration of an embodiment of a detection device of this invention.

FIG. 4 is an illustration of an embodiment of a detection device of this invention.

FIG. 5 is an illustration of an embodiment of a detection device of this invention.

FIG. 6 is an illustration of an embodiment of a detection device of this invention.

### DETAILED DESCRIPTION OF THE PREFERRED EMBODIMENTS

During bleeding, broken red blood cells release components of occult blood including hemoglobin, hemoglobin-haptoglobin-complex, transferrin and albumin. Hemoglobin can be destroyed by acid and other environmental factors in the digestive system while the other components of occult blood such as hemoglobin-haptoglobin-complex, transferrin and albumin are not as easily destroyed as they have a different interaction with the environment factors in the digestive tract. Testing only for the presence of hemoglobin as an indicator of the presence of occult blood can produce false negatives, especially for samples or specimens from patients with relatively upper digestive tract bleeding. Testing for the presence of hemoglobin-haptoglobin-complex, transferrin or albumin as part of the occult blood test can avoid or reduce the false negatives rate resulting from relatively upper digestive tract bleeding. Therefore, the presently preferred embodiments of this invention, the One Step OB (Occult Blood) Combo Test, is a simple one step immuno-chromatographic assay that can provide for the detection of hemoglobin as well as the other components of occult blood.

The components of occult blood that may be tested are also referred to herein as antigens. Specific components of occult blood, i.e., specific antigens may be referred to their name, i.e., hemoglobin, haptoglobin-hemoglobin complex, transferrin, and albumin. The antibodies to the antigens may be referred to herein as test antibodies. Specific antibodies to a specific antigen may be referred to by name, i.e., anti-hemoglobin antibodies, anti-hemoglobin-haptoglobin complex antibodies, anti-transferrin antibodies, and anti-albumin antibodies.

The preferred embodiments are devices and methods that can provide for the testing of the presence of two or more of the following components of occult blood (antigens): hemoglobin, hemoglobin-haptoglobin-complex, transferrin and albumin. Other preferred embodiments of this invention can provide for the detection for the presence of one or more of the following components of occult blood: hemoglobin-haptoglobin-complex, transferrin and albumin. The use of methods of this invention or devices that are embodiments of this invention can significantly increase the accuracy and reduce the occurrence of false negative results.

The One Step OB Combo Test employs a unique combination of monoclonal and/or polyclonal antibodies to selectively detect one or more components of occult blood: human hemoglobin, hemoglobin-haptoglobin-complex, transferrin and albumin in samples with a high degree of sensitivity. In one embodiment, the detection sensitivity for hemoglobin is 10 ng/ml, hemoglobin-haptoglobin-complex is 10 ng/ml, transferrin is 1 ng/ml, and albumin is 1 ng/ml.

The preferred embodiments of a detection device for the detection of occult blood using the One Step OB Combo Test is a one-step lateral flow chromatographic immunoassay having two or more of the following four test areas to test for the respective components of occult blood:

a hemoglobin test area having anti-hemoglobin antibodies to detect the presence of hemoglobin;

a haptoglobin test area having anti-hemoglobin-haptoglobin complex antibodies to detect the presence of hemoglobin-haptoglobin-complex;

a transferrin test area having anti-transferrin antibodies to detect the presence of transferrin; and

an albumin test area having anti-albumin antibodies to detect the presence of albumin.

Other preferred embodiments of a detection device for the detection of occult blood using the One Step OB Combo Test is a one-step lateral flow chromatographic immunoassay having one or more of the following three test areas to test for the respective components of occult blood:

a haptoglobin test area having anti-hemoglobin-haptoglobin complex antibodies to detect the presence of hemoglobin-haptoglobin-complex;

a transferrin test area having anti-transferrin antibodies to detect the presence of transferrin; and

an albumin test area having anti-albumin antibodies to detect the presence of albumin.

The test antibodies for a specific test area can be any animal antibodies. Examples of animal antibodies that can be used for the test areas are goat antibodies, mouse antibodies, and rabbit antibodies. For example, if mouse antibodies are used in a hemoglobin test area, then, the antibodies in that area would be mouse anti-hemoglobin antibodies.

When a test sample is inserted or flows into a test area with a specific antibody, if the corresponding antigen to the specific antibody is present in the test sample, it will bind with the antigen to form an antigen-antibody complex. Presence of that antigen-antibody complex will indicate the presence of that specific antigen in the test sample.

The number and identity of the antigens to be tested, that is, the number of different test areas in a device, are dependent on the criterion used to determine the presence of occult blood and the desired accuracy of the testing result. For example, if a positive result for occult blood, i.e., a determination that occult blood is present in a test sample, requires only that one of the above-four stated components be present, the device can have from one to four test areas, each area testing for one of the antigen. The presence of an antigen in any one of the test area will indicate a positive result. However, having only one test area to detect the presence of hemoglobin can produce false negatives, due to the possible destruction of hemoglobin in the upper digestive tract. Therefore, testing for the presence of the other antigens in a test sample by increasing the number of test areas will reduce the number of false negatives and increase the accuracy of the result.

If two or more test areas are used, but the presence of any one antigen is sufficient for a positive determination of the presence of occult blood, then the different test areas can physically occupy the same area in a device. In one preferred embodiment where the presence of either hemoglobin or hemoglobin-haptoglobin complex is used to determine the presence of occult blood, the device can have one test region that contains both the hemoglobin test area and the haptoglobin-hemoglobin test area.

If the presence of occult blood requires the presence of two or more components of occult blood in the test sample, then two or more test areas that are physically separate can be used for the testing of the test sample.

The test areas can be of any shape or size. In some preferred embodiments, it can be in the form of a test line.

In order to easily determine the occurrence of antigen-antibodies reaction for an antigen in the test areas, i.e., in order to easily identify the presence of antigens in a test sample, preferred embodiments also have a conjugate reagent area containing a conjugate reagent that is conjugated with the test antibodies. Examples of conjugate reagents that can be used are color particles such as latex, latex particles, gold particles, or colloidal gold. This conjugate reagent area can be placed between the point of insertion, i.e., the point where the test sample is inserted onto the device, and the test area or areas.

The quantity of test antibodies in each of the test area is adjusted such that the test area appears in a pre-determined color when the concentration of the component to be detected is at or above a pre-determined sensitivity level. In preferred embodiments where colloidal gold is the conjugate reagent, the quantity of anti-hemoglobin antibodies in the hemoglobin test area can be adjusted such that this area will appear burgundy when the concentration of the hemoglobin in the test sample is at or above a pre-determined level such as 10 ng/ml. Similarly, the quantity of the anti-hemoglobin-haptoglobin complex antibodies in the haptoglobin test area can be adjusted such that this area will appear burgundy when the concentration of the hemoglobin-haptoglobin-complex in the test sample is at or above a pre-determined level such as 10 ng/ml, the quantity of anti-transferrin antibodies in the transferrin test area can be adjusted such that this area will appear burgundy when the concentration of the transferrin in the test sample is at or above a pre-determined level such as 1 ng/ml, and the quantity of anti-albumin antibodies in the albumin test area can be adjusted such that this area will appear burgundy when the concentration of albumin in the test sample is at or above a pre-determined level such as 1 ng/ml.

In order to ensure that a sufficient quantity of the test sample is being introduced to the test device, preferred embodiments also have one or more control areas for quality control. The control area may have any animal antibodies, referred to herein as control antibodies. The control animal antibodies can be any animal antibodies to the test antibodies as long as it is reactive to the conjugate reagent. It is preferable that the type of animal used for the control antibodies not be the same as the type of animal used for the test antibodies. If the test antibodies are mouse antibodies, the control antibodies can be rabbit anti-mouse antibodies, goat anti-mouse antibodies. Similarly, if the antibodies in one test area are mouse antibodies while the antibodies used in another test area are rabbit antibodies, then, the control antibodies may be goat anti-mouse antibodies and goat anti-rabbit antibodies. For embodiments with one or more control areas, the conjugate reagent area can also contain the conjugate reagent conjugated with the control antibodies.

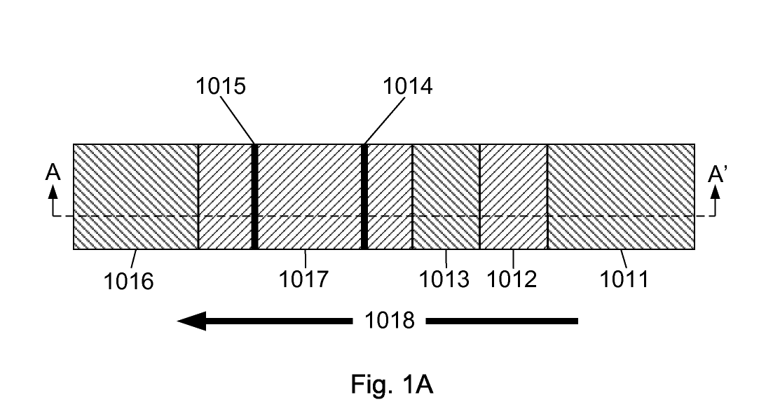

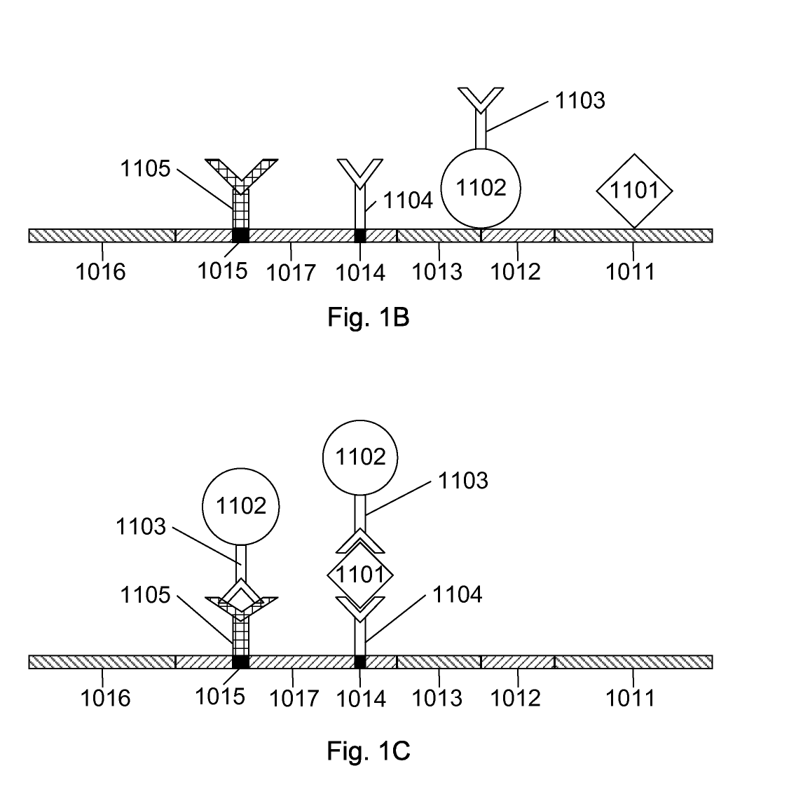

In some preferred embodiments, the test areas for the different antigens to be tested are placed on a strip such that the test sample can diffuse or migrate from the point of insertion to the test areas. FIG. 1 (a) is an example of an embodiment of this invention that is a strip having a specimen area as the point of insertion (1011), an optional separation area (1012), a conjugate reagent area (1013), a test line that can contain one or more test areas (1014), a control area in the form of a control line (1015), and an absorption area (1016). The test line and the control line can be on a membrane (1017). The arrow underneath the illustrated embodiment (1018) indicates the flow or diffusion direction of the test sample.

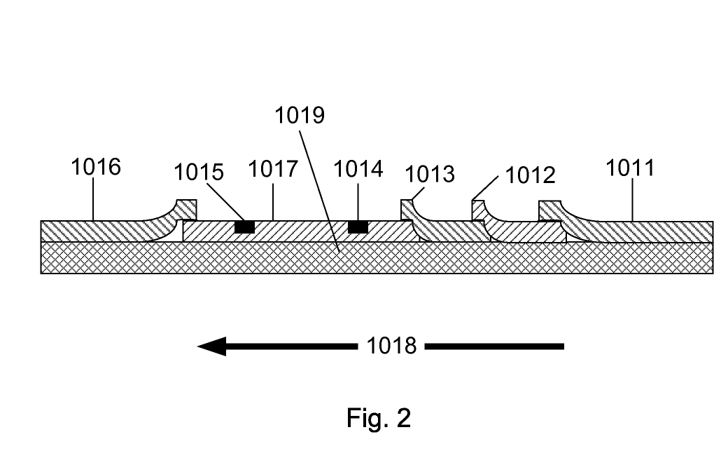

FIG. 2 is the A-A′ cross section of the embodiments illustrated in FIG. 1a. The point of insertion (1011), an optional separation area (1012), conjugate reagent area (1013), test line (1014), control line (1015), and an absorption area (1016) are placed on top of a backing strip such as a PVC backing (1019). The materials for the different areas are materials that would allow the diffusion or flow of the test sample from the specimen area through the separation area, the conjugate reagent area, the test line, the control line to the absorption area. For example, the test sample, together with complexes that are generating during its flow can be transported by capillary action supported by the absorption area. The test line can be a portion of a membrane (1017) such as a nitrocellulose membrane that contains the test antibodies to the antigen or antigens to be tested. These antibodies are fixed or immobilized to the membrane. Similarly, the control line can be another portion of the membrane (1017) that contains control antibodies that are fixed are immobilized to the membrane.

The separation pad does not contain any conjugate reagent or antibodies. The conjugate reagent area contains a conjugate reagent that is conjugated with the test antibodies. It may also contain the conjugate reagent that is conjugated with the control antibodies. The absorption area is an area where the test sample is absorbed after it has flowed or diffused through the strip.

FIGS. 1(b) and 1(c) also show the A-A′ cross section of the embodiments illustrated in FIG. 1a and illustrates the state of the conjugate reagent and the test and control antibodies on the strip before and after a test sample containing a specific antigen to be tested has diffused through the strip. In FIG. 1(b), just after test sample has been inserted onto the specimen area and before the test sample has flowed into the strip, the specimen area contains the test sample with the antigen (1101). The conjugate reagent area contains the conjugate reagent (1102) such as colloidal gold that is conjugated with and attached to test antibodies and/or control antibodies (1103). The test area contains the immobilized test antibodies corresponding to the antigen to be tested (1104) while the control area contains the immobilized control antibodies (1105). FIG. 1(c) illustrates what happens after a test sample containing the antigen to be tested has passed through the strip. As the test sample flows or diffuses through the conjugate reagent area, the antigen forms a conjugate reagent-antibody-antigen complex. The test samples then carries this conjugate reagent-antigen-antibody complex, well as conjugate reagents conjugated with test antibodies or control antibodies to the test area. At the test area, a new complex, the conjugate reagent-antibody-antigen-antibody complex is formed by the combination of the antibody-antigen-conjugate reagent complex with the immobilized test antibodies. The antigen in this new complex (1101) is encased by the immobilized test antibody (1104) and the test antibody (1103) that is conjugated with the conjugate reagent (1102) and is immobilized at the test line. If a sufficient quantity of these complexes is immobilized, the test line or area will display the color of the conjugate reagent indicating a positive result. For example, if the conjugate reagent is colloidal gold, the test line will turn burgundy if a pre-determined quantity of the antibody-antigen-conjugate reagent-antibody complex is immobilized at the test line. If an insufficient quantity of these complexes are formed an immobilized, the test line will not change to the color of the conjugate reagent, indicating a negative result.

The remainder of the test sample as well as some conjugate reagent conjugated with the test antibodies or control antibodies continues to flow down the strip. At the control area (1015), the conjugate reagent (1102) that is conjugated with the test or control antibodies (1103) forms a complex with the immobilized control antibodies (1105). If a sufficient quantity of these complexes is formed, then the control would change to the color of the conjugating reagent indicating that a sufficient quantity of the test sample has flowed to the control area and validating the results of the test. The remainder of the test sample then continues to flow down the strip and is absorbed at the absorption pad.

The above description describes the testing of a single antigen. Similar embodiments can contain a test line with more than one test area. That is, the test line can contain immobilized antibodies for the several antigens to be tested. The conjugate reagent area would then contain conjugate reagents that are conjugated with the antibodies to each of the antigens to be tested and/or control antibodies. At the test line, for each antigen to be tested, if the antigen is present, then the antigen and its corresponding antibody will form conjugate reagent-antibody-antigen-antibody complexes that are immobilized at the test line. The test line would change to the color of the conjugate reagent when a pre-determined number of complexes of these complexes from the different antigens to be tested are formed. For example, if the antigens to be tested are hemoglobin and hemoglobin-haptoglobin-complex, then the test line would contain the hemoglobin and haptoglobin test areas that contain the immobilized anti-hemoglobin antibodies and anti-hemoglobin-haptoglobin complex antibodies. This test are would change color whenever the total number of conjugate reagent-anti-hemoglobin antibodies-hemoglobin antigen-anti-hemoglobin antibody complexes and conjugate reagent-anti-hemoglobin-haptoglobin complex antibodies-hemoglobin-haptoglobin-complex antigen-anti-hemoglobin-haptoglobin complex antibodies reaches a pre-determined quantity.

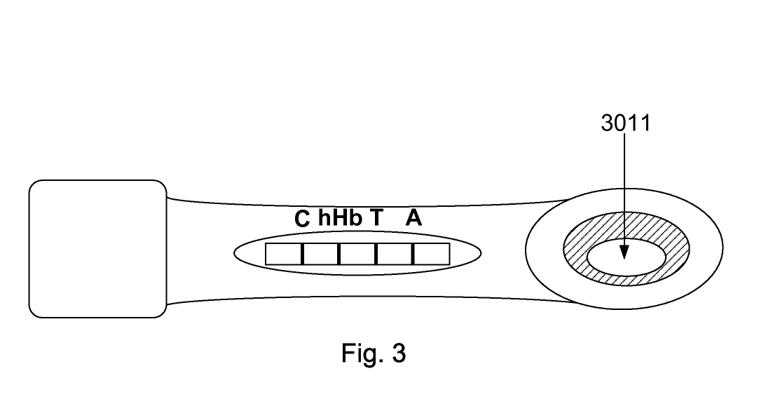

Another variation of the above described embodiments can be a test strip that comprises more than one test line where each test line contains a test area. FIG. 3 is an illustration of these embodiments for the testing of three antigens, hemoglobin, transferrin, and albumin where the test strip is encased in a cassette and the three test lines and a control line in this embodiment are visible through a window underneath the characters hHb, T, A, and C respectively. The point of insertion (3011) is a well at one end of the cassette. Droplets of test sample can be inserted into the well. The fluid placed in the well can then flow, diffuse or migrate through the well onto the strip and then to the different test areas and the control area. If the control area changes color indicating the test results are valid, the interpretation of whether there is occult blood will depend on the decision criteria set by the interpreter. If none of the test areas change color, then no occult blood is found with this test. If one or more of the test area changes color, this test indicates that one or more components of occult blood are present. An interpreter can decide that one or more of the test areas changing color indicates the presence of occult blood. To reduce the possibility of any false negatives, only one test area need change color for an interpretation of a presence of occult blood.

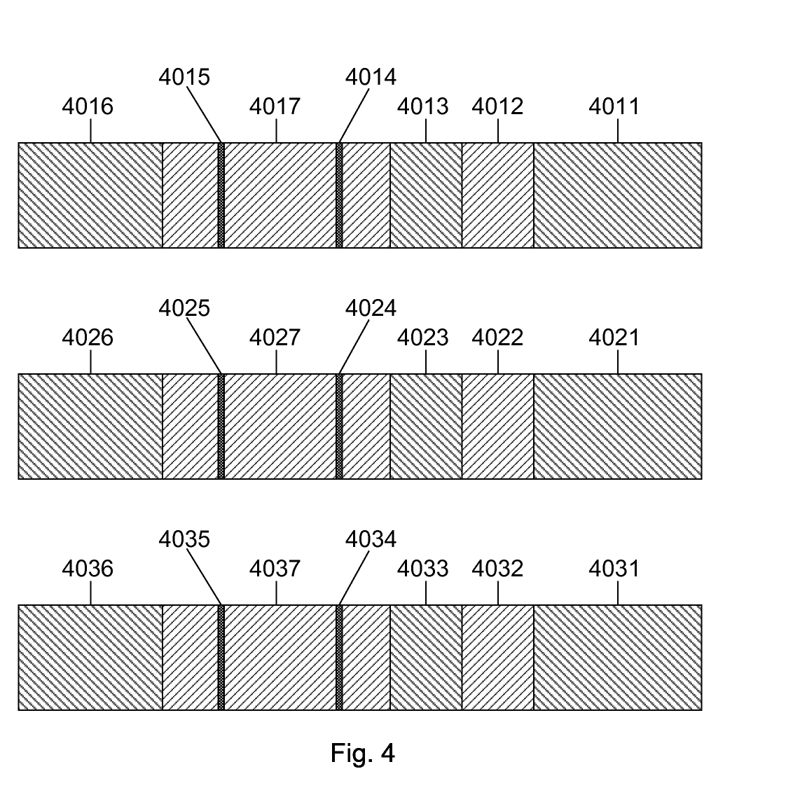

FIG. 4 illustrates another preferred embodiment. This device comprises of 3 test strips, each of which contains a strip that would allow the testing of 3 separate antigens, one on each strip. Each of the strip has an insertion area (4011, 4021, and 4031), a separation area (4012, 4022, and 4032), a conjugate reagent area (4013, 4023, and 4033), a test area (4014, 4024, and 4034) that is on or part of a membrane (4017, 4027, and 4037), a control area (4015, 4025, and 4035) that is on or part of a membrane (4017, 4027, and 4037), and an absorption area (4016, 4026, and 4036). In other variation of this embodiment, any one of the strips can have one or more test area to test for more than one antigen.

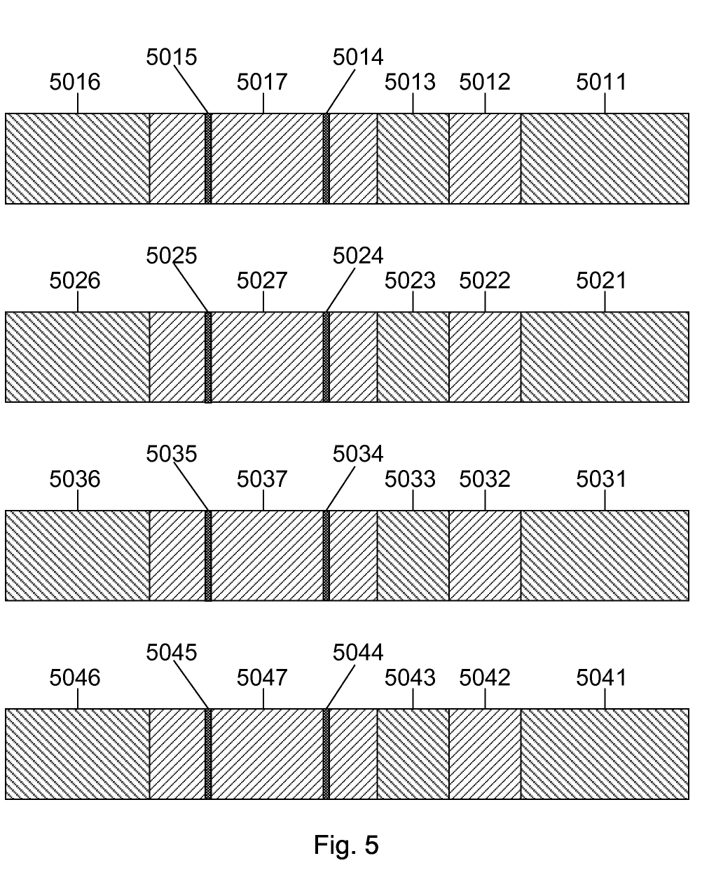

FIG. 5 illustrates a fourth preferred embodiment that contains 4 strips, one for each of the component of occult blood to be tested. Each strip contains an insertion area (5011, 5021, 5031, and 5041), a separation area (5012, 5022, 5032, and 5042), a conjugate reagent area (5013, 5023, 5033, and 5043), a test area (5014, 5024, 5034, and 5044) that is on or part of a membrane (5017, 5027, 5037, and 5047), a control area (5015, 5025, 5035, and 5045 that is on or part of a membrane (5017, 5027, 5037, and 5047), and an absorption area (5016, 5026, 5036 and 5046).

Each of the conjugate reagent areas in the above embodiments can contain a conjugate reagent that is conjugated with the antibodies to the components of the blood to be tested and/or the antibodies used for the control line. Each test line contains the antibodies to the antigens to be tested for that test area or line. Each control area contains control antibodies.

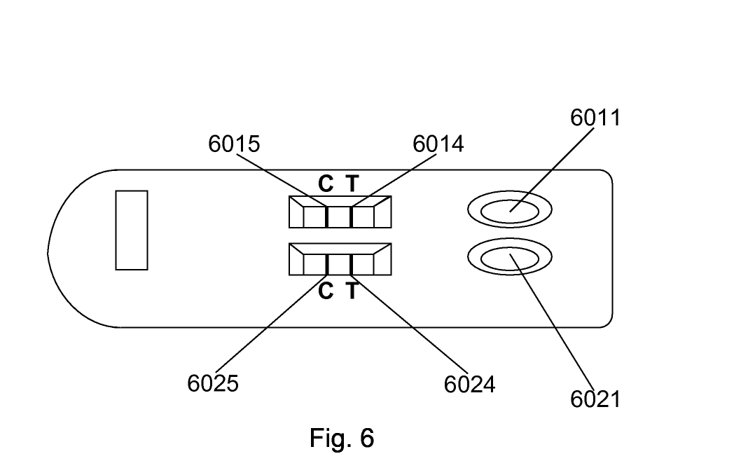

Embodiments with one or more strips each containing one or more test areas or lines can be encased in a cassette with one or more wells are the points of insertion and windows for each test line or area. FIG. 6 is an illustration of a cassette containing two strips, each strip having a well (6011 and 6021), a test line (6014 and 6024), and a control line (6015 and 6025).

The preferred methods for detection for the presence of occult blood in a sample or specimen such as a fecal sample by detecting the presence of two or more components of occult blood includes the steps of:

collecting a sample;

placing the sample in a sample collection device such as a fecal collection tube;

preparing said sample for testing, if necessary, by mixing it with a pre-determined quantity of fluid to form a fluid sample. The prepared sample, or the sample that can be used without preparation for the detection is referred to herein as a test sample. The mixing can be conducted by shaking the specimen with a pre-determined fluid in the sample collection device;

bringing the prepared test sample in its sample collection device to room temperature, if needed;

exposing the test sample to two or more antibodies to the components of occult blood selected from the group consisting of: anti-hemoglobin antibodies, anti-hemoglobin-haptoglobin-complex antibodies, anti-transferrin antibodies, and anti-albumin antibodies for a pre-determined period of time;

determining the level of reactions between said selected two or more antibodies to the components of occult blood and their corresponding components of occult blood that may be in the test sample; and

deciding whether occult blood is present in the test sample as a function of the level reactions. This deciding step may include the indicating of the presence of occult blood as a function of the level of reactions.

The number and identity of the components of occult blood to be tested are dependent on the criterion used to decide the presence of occult blood and the desired accuracy of the testing result. For example, if a positive result for occult blood, a decision that occult blood is present in a test sample, requires that two of the components be present, then testing for the presence of two out of the four components is sufficient. If the criterion to decide for the presence of occult blood only requires that one of the above-four stated components is present, the test of only one of the four components may be sufficient. Therefore, variations of the above described method test detect for the presence of one or more components by replacing the exposing step in the above described method by the following step:

exposing said test sample to one or more antibodies to the components of occult blood selected from the group consisting of: anti-hemoglobin-haptoglobin-complex antibodies, anti-transferrin antibodies, and anti-albumin antibodies for a pre-determined period of time.

Detecting only for the presence of one component of occult blood can produce false negatives, if the quantity of that component is reduced during the digestive process. Each of the proteins that are the components of occult blood: hemoglobin, hemoglobin-haptoglobin-complex, transferrin and albumin, interact with the environment of the digestive tract differently. When using the above described methods, it is preferred to couple the detection of hemoglobin with the detection at least one other component of occult blood that will not be destroyed in the digestive process. This will greatly reduce the number of false negatives, especially when testing for relatively upper digestive tract bleeding where more environmental and occult blood interactions can occur.

The exposure of said fluid test sample to the antibodies of the components to the occult blood to be detected can occur in the corresponding test area for that component in the devices that are embodiments of this invention. If a component of the occult blood is present, the reaction in the test area for that component will result in complexes of antigen-antibody for that component.

In order to easily determine the level of antigen-antibodies complexes formed for a component, i.e., in order to easily identify the presence of a component in the test sample is above a pre-determined level for that component, the test sample is also exposed to a conjugate reagent that is conjugated with the test antibodies. Colloidal gold and latex are examples of conjugate reagent with antibodies. As described previously, a change of color for a test area containing the antibodies to the component of the occult blood to the color of the conjugate reagent used will indicate the presence of the component. In the reverse, a failure for the test area of a component to change color to the color of the conjugate reagent will indicate the absence of the substance above the pre-determined level.

In preferred embodiments, the quantity of test antibodies to a specific antigen that a test sample is exposed to is adjusted such that the test area where the exposure occurs will appear burgundy when the concentration of the specific antigen is at or above a pre-determined level. In preferred embodiments, that level is 10 ng/ml for hemoglobin, 10 ng/ml for hemoglobin-haptoglobin complex, 1 ng/ml for transferring, and 1 ng/ml for albumin.

In order to ensure that an adequate quantity of the test sample is exposed to the test antibodies in each of the test areas to generate an observable result, the test sample is also exposed to control antibodies in a control area. Control areas such as the control areas in preferred embodiments of the detection device described previously in this document could be used. The test sample flows, diffuses, or migrates through the control area and results are considered invalid unless the control area changes color. This may happen if an insufficient quantity of the test sample is used or placed onto the device.

As described earlier, an interpreter can set the criteria for deciding whether there is occult blood. In preferred embodiments, the presence of any one of the components of occult blood above the pre-determined level will indicate the presence of occult blood.

Dietary restrictions are not necessary for detections that use the devices that are embodiments of this invention or methods of this invention. However, alcohol, aspirin and other medication than can cause gastrointestinal irritation and result in occult bleeding should be discontinued at least 48 hours prior to specimen collection to limit the false positives for colorectal cancer or polyps. In addition, to avoid false positives resulting from bleeding from known causes, specimen should not be collected during or within three days of a menstrual period, or if the patient suffers from bleeding hemorrhoids or blood in the urine.

For the detection of occult blood that uses the devices that are preferred embodiments of this invention or that uses methods of this invention, only a small amount of fecal specimen or sample need to be collected and used. Different sample collection methods or device can be used, including currently used devices and methods of collection. For example, a fecal specimen, free from contamination of toilet water, can be collected on a toilet paper or a clean container. To prepare the sample for testing, a pre-determined fluid such as a sodium salt solution or a buffer solution is added to the sample, either immediately after the collection of the sample or just before testing. This fluid can be added to the sample in the sample collection device with the collected sample and the device is shaken well. Optimally, the collected sample can be stored at room temperature (below 30° C.) for 7 days or refrigerated at 4 to 8° C. for 14 days.

For testing, one or more drops of the test sample, is dispensed onto each of the test areas such that the test sample is exposed to the antibodies in each of the test areas. For example, in the detection device shown in FIG. 3, a preferred embodiment comprising of a strip encased in a cassette, the sample well formed by a membrane is placed on a strip at one end of the membrane. The control, hemoglobin test, transferrin, and albumin test areas, are under the cassette labeled “C”, hHb”, “T”, and “A” respectively. The droplets of test sample can be inserted onto the well. The fluid placed in the well then migrates through the membrane of the well onto the different test areas and the control area. If the test sample is at room temperature of between 15° C. to 30° C., preferably, the test sample should be allowed to react for 10 to 15 minutes before determining whether there is any color change in the any of the test and control areas. If a positive result, that is, a color change, is established at a test or control area in between 10 and 15 minutes, the results should not change. However, to prevent any incorrect results, the determination of whether there is any color change should not be conducted after more than 15 minutes of reaction between the test sample and the antibodies in the test area. If the temperature of the specimen is significantly different than room temperature, then the time allowed for the reaction before the determination of any color change should be adjusted. For example, if the room temperature and therefore the temperature of the specimen is significantly lower than 15° C., the time for the reacting step may have to be increased.

The presence of occult blood in stools may result from other conditions other than colorectal bleeding such as hemorrhoids, blood in urine, or stomach irritations. Embodiments of this invention are simple and inexpensive and designed to produce a rapid result. If a positive result is obtained, additional diagnostic procedures should be performed to determine the cause and source of the occult blood. Moreover, negative results do not exclude colorectal polyps or cancer. Some colorectal polyps and cancer may not bleed at their early stages or they may bleed intermittently. Even there is bleeding from colorectal polyps or cancer, negative results may still occur. The sample collected may also fail to capture the occult blood if the occult blood is not distributed evenly throughout the feces. The sample may have been collected when bleeding was not occurring.

While the present invention has been described with reference to certain preferred embodiments, it is to be understood that the present invention is not limited to such specific embodiments. Rather, it is the inventor's contention that the invention be understood and construed in its broadest meaning as reflected by the following claims. Thus, these claims are to be understood as incorporating not only the preferred embodiments described herein but all those other and further alterations and modifications as would be apparent to those of ordinary skilled in the art.

We Claim:

1. A method for the detection of occult blood in a test sample comprising the steps of:
exposing said test sample to two or more antibodies to components of occult blood, said antibodies are selected from the group consisting of: anti-hemoglobin antibodies, anti-hemoglobin-haptoglobin-complex antibodies, anti-transferrin antibodies, and anti-albumin antibodies;
determining the level of reactions between said selected two or more antibodies to the components of occult blood and their corresponding components of occult blood that may be in the test sample; and
deciding on the presence of occult blood as a function of the level of reactions.

2. The method of claim 1 wherein in said exposing step said test sample is also exposed to a conjugate reagent.

3. The method of claim 1 wherein said deciding step comprising the substep of indicating the presence of occult blood as a function of the level of reactions.

4. The method of claim 2 wherein said conjugate reagent is colloidal gold or latex.

5. The method of claim 2 wherein said deciding step comprising the substep of indicating the presence of occult blood as a function of the level of reactions.

6. A method for the detection of occult blood in a test sample comprising the steps of:
exposing said test sample to one or more antibodies to components of occult blood, said antibodies selected from the group consisting of: anti-hemoglobin-haptoglobin-complex antibodies, anti-transferrin antibodies, and anti-albumin antibodies;
determining the level of reactions between said selected one or more antibodies to the components of occult blood and their corresponding components of occult blood that may be in the test sample; and
deciding on the presence of occult blood as a function of the level of reactions.

7. The method of claim 6 wherein in said exposing step, said test sample is also exposed to a conjugate reagent.

8. The method of claim 6 wherein said deciding step comprising the substep of indicating the presence of occult blood as a function of the level of reactions.

9. The method of claim 7 wherein said deciding step comprising the substep of indicating the presence of occult blood as a function of the level of reactions.

10. The method of claim 7 wherein said conjugate reagent is colloidal gold or latex.

11. A detection device for the detection of occult blood, comprising two or more of the following test areas for the detection of the components of occult blood: a hemoglobin test area, a haptoglobin test area, a transferrin test area, and, an albumin test area; and wherein
said hemoglobin test area having anti-hemoglobin antibodies;
said haptoglobin test area having anti-hemoglobin-haptoglobin-complex antibodies;
said transferrin test area having anti-transferrin antibodies; and
said albumin test area having anti-albumin antibodies.

12. The detection device of claim 11 also comprising a conjugate reagent area having a conjugate agent.

13. The detection device of claim 12 wherein said conjugate reagent is colloidal gold or latex.

14. The detection device of claim 12 wherein said conjugate reagent area also having two or more antibodies to the components of occult blood selected from the group consisting of: anti-hemoglobin antibodies, anti-hemoglobin-haptoglobin-complex antibodies, anti-transferrin antibodies, and anti-albumin antibodies

15. The detection device of claim 14 wherein said conjugate reagent is colloidal gold or latex.

16. A detection device for the detection of occult blood, comprising one or more of the following test areas for the detection of the components of occult blood: a haptoglobin test area, a transferrin test area, and, an albumin test area; and wherein
said haptoglobin test area having anti-hemoglobin-haptoglobin-complex antibodies;
said transferrin test area having anti-transferrin antibodies; and
said albumin test area having anti-albumin antibodies.

17. The detection device of claim 16 also comprising a conjugate reagent area having a conjugate agent.

18. The detection device of claim 17 wherein said conjugate reagent is colloidal gold or latex.

19. The detection device of claim 17 wherein said conjugate reagent area also having one or more antibodies to the components of occult blood selected from the group consisting of: anti-hemoglobin-haptoglobin-complex antibodies, anti-transferrin antibodies, and anti-albumin antibodies

20. The detection device of claim 19 wherein said conjugate reagent is colloidal gold or latex.
